(19) United States
(12) Patent Application Publication
(10) Pub. No.: US 2012/0178123 A1
(43) Pub. Date: Jul. 12, 2012

(54) ENHANCED LIPID PRODUCTION FROM ALGAE

(76) Inventor: Barry Rosen, Longwood, FL (US)

(21) Appl. No.: 13/496,415

(22) PCT Filed: Sep. 16, 2010
(86) PCT No.: PCT/US10/49145

§ 371 Date: Mar. 15, 2012

Related U.S. Application Data
(60) Provisional application No. 61/242,915, filed on Sep. 16, 2009.

(51) Int. Cl. C12P 7/64; C12M 1/36; C12P 39/00

(52) U.S. Cl. 435/42; 435/134; 435/286.1

(57) ABSTRACT

A method for stimulating enhanced lipid accumulation by algae includes growing algae in a bioreactor medium including nutrients. The algae have an average lipid content that averages a first % of total cell biomass. A stress inducing environmental condition is initiated that keeps the algae alive, stops cell reproduction, and induces the algae to accumulate additional lipids resulting in a second average lipid content that is at least 50% more than the first %. The method can include measuring a lipid concentration of the algae while under the stress inducing environmental condition, harvesting lipids from more than 50% but not all of the algae when the lipid concentration is above a predetermined lipid limit, adding fresh medium to the bioreactor medium having algae not involved in the harvesting therein, and repeating the method, wherein the algae not involved in harvesting serves as a source for new algae growth.

### CROSS REFERENCE TO RELATED APPLICATION

This application is a national stage application that claims priority to PCT/US/2010/049145 filed Sep. 16, 2010, which claims priority to provisional patent application 61/242,915 filed Sep. 16, 2009, both of which are incorporated herein in their entireties.

### FIELD OF THE INVENTION

Disclosed embodiments relate to lipid production from algae.

### BACKGROUND

Various attempts have been made to develop biofuels from non-petroleum sources. For example, an effort has been made to develop ethanol from plant materials, primarily from corn grain. However, the resulting impact on corn and food prices suggests that there are limits to how much further such production is feasible.

Other technologies have been developed to produce biodiesel from plant sources. Many different irrigated crops, such as soybean, rapeseed, palm and sunflower, can be used to produce biodiesel. Current biodiesel production often utilize some form of transesterification process, wherein triglycerides or other starting materials undergo an alkali or acid catalyzed transesterification reaction between the fatty acid component of the triglyceride and a low molecular weight alcohol, such as methanol. Glycerol is released as a byproduct of transesterification and fatty acid methyl esters are produced. Such processes may be operated in either a batch or continuous mode. However, it is currently necessary to first separate the triglycerides or other source material from the bulk plant matter before the transesterification reaction can proceed.

Alternatives to increase biofuels production capacity have been proposed, such as conversion to cellulosic ethanol production, utilizing wood, switchgrass or other non-food starting materials. However, cellulosic ethanol technology, has not yet been developed to the point of full commercial scale production and the time required to reach that point remains uncertain. Other proposals have involved biofuel crop production on marginal or idle land, such as the Conservation Reserve Program (CRP) acreage. Such proposals ignore the practical difficulties of obtaining water supplies to grow such crops, requirements for fertilizer input, low productivity of marginal land.

Another alternative source of biofuels production has been proposed for algal culture systems. One obstacle to algal culture is because algae are protected by a tough cell wall. That wall must be cracked, typically an energy-expensive process, to extract the lipids which can be converted to biodiesel. The National Renewable Energy Laboratory (NREL) in Golden, Colo. over a decade and more than $25 million on an Aquatic Species program that focused on extracting biodiesel from unusually productive species of algae. NREL scientists demonstrated oil production rates two hundred times greater per acre than achievable with fuel production from soybean farming. However, the open pond system utilized by NREL was susceptible to invasion by contaminating algae, bacteria or algal-consuming organisms and algal productivity was adversely impacted by fluctuating environmental temperature and solar radiation. Further, in a pond type of system the light penetration depth into dense algal cultures results in only a limited band of photosynthetic productivity, with the majority of algae being shaded by overlying organisms.

### SUMMARY

Methods for enhanced lipid production from algae are disclosed that enable continuous lipid production without sacrifice of all the cells. Lipid extraction as disclosed herein is inherently more efficient as compared to conventional lipid production which involves cell sacrifice/lysis of all cells because by keeping the algae cells alive during lipid enhancement allows the per cell lipid output to be far more as compared to a single amount obtained from conventional methods.

Disclosed embodiments are based on the Inventor's recognition of several significant phenomena that can be present when algae encounter stress inducing environmental conditions that are sufficiently adverse for the natural processes of the algae to trigger a cellular shut down defined herein as the ending of logarithmic growth, but not adverse enough to kill most of the algae, with typically no measurable amount of the algae killed by the stress inducing environmental conditions. One of the key results of the shut down processes disclosed herein is the degradation of the internal membranes of the algae, such as those in the chloroplast, is the production of more lipids induced by stressing the algae, and the subsequent rearranging of these membrane lipids into a centralized mass of neutral lipid.

Under conventional growth stimulating environmental conditions, the redistribution of lipids in algae occurs to a limited extent as part of the aging process of the algae. The methods described herein forces the algae to substantially increase their lipid content, defined as at least a 50% increase and typically 100% (i.e., double) or more of their lipid content over the lipid content referred to herein as the “first %” that is present during normal growing conditions, and inducement of a change from the normal distribution of lipids in the algae into a centralized lipid mass. In one embodiment the first % is at least 25% and the second concentration is at least 50%.

### BRIEF DESCRIPTION OF THE DRAWINGS

FIG. 1 is a flow chart for an example method for stimulating increased lipid production from algae, according to an embodiment of the invention.

FIG. 2 is a block diagram of an exemplary bioreactor system for generating enhanced lipid production from algae, including a dynamic control system, according to an embodiment of the invention.

### DETAILED DESCRIPTION

Several aspects of the invention are described below with reference to example applications for illustration. It should be understood that numerous specific details, relationships, and methods are set forth to provide a full understanding of embodiments of the invention. One having ordinary skill in the relevant art, however, will readily recognize that embodiments of the invention can be practiced without one or more of the specific details or with other methods. In other instances, well-known structures or operations are not shown in detail to avoid obscuring inventive details. Embodiments of invention are not limited by the illustrated ordering of acts or events, as some acts may occur in different orders and/or concurrently with other acts or events. Furthermore, not all illustrated acts or events are required to implement a methodology in accordance with embodiments of the invention.

Disclosed embodiments relate to increasing lipid production from live algae by providing certain environmental cues, such as nitrogen-deficiency, that triggers the degradation of their membranes and enhanced lipid synthesis that results in enhanced lipid accumulation in algal cells. Although not needed to practice embodiments of the Invention, it is believed that algae perform enhanced lipid accumulation in response to stress inducing environmental conditions disclosed herein that functions to conserve lipids for future assembly back into functioning organelles, such as chloroplasts, once environmental conditions support normal growth and reproduction.

One aspect disclosed herein is the addition of one or more compounds in an effective concentration that actively stops cell growth just prior to cells entering the stationary phase of growth, without cell death, which allows cells to enlarge and accumulate substantially more lipid than would occur naturally under conventional growing conditions. As known in the art, algae are a very diverse and simple group of aquatic plant that are widespread across the world. Algae can vary in form from Eukaryote to Bacteria, and are spread across the kingdoms Plantae, Protista, and Protozoa. All forms can generally generate excess lipids based on methods disclosed herein, which can be converted to various renewable fuels, such as biodiesel. In some embodiments, the algae types used for culture are photosynthetic Plantae algae, although the skilled artisan will realize that alternative algal types may be utilized in the practice of the disclosed methods.

The algae are typically selected for their high lipid accumulating ability and efficient growth under a variety of conditions. It has been found that both freshwater and marine algae species can be induced to accumulate excess lipids. Moreover, various genetic engineering strategies can be further employed to increase total lipid production and also vary the chemical composition of lipids produced by the algae strain, including targeting saturation/desaturation of hydrocarbons and varying the carbon chain length.

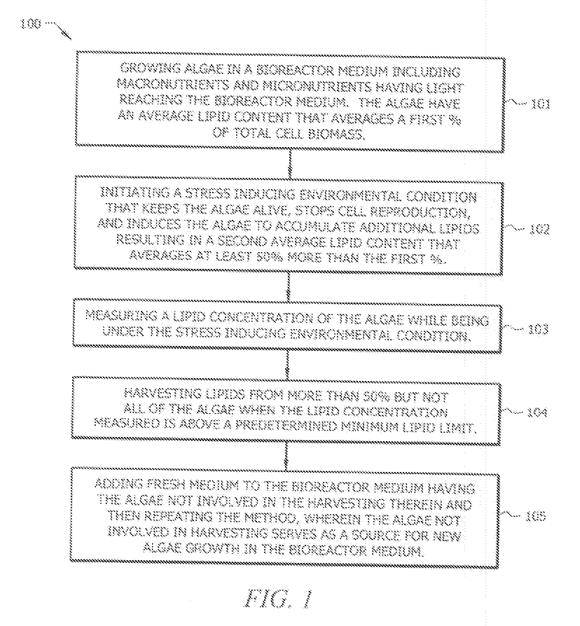

FIG. 1 is a flow chart for an example method 100 for stimulating enhanced lipid production from algae, according to an embodiment of the invention. Methods disclosed herein can increase the lipid content in the algae by at least 50% of the total cell weight/biomass, and typically by 100% (a doubling), or more. For example, the lipid content due to use of disclosed adverse environmental conditions that induces stress can increase the lipid content in a particular species of algae from an average of 25% to 50% of the total cell biomass. The gain in lipid content is primarily a function of losses from both the carbohydrate and protein portion of the cells.

Step 101 comprises growing algae in a bioreactor medium including nutrients (macronutrients and micronutrients) having light reaching the medium. The algae reach an average lipid content that averages a first % of total cell biomass under the conditions provided in Step 101. Step 102 comprises initiating at least one stress inducing environmental condition that keeps the algae alive, stops cell reproduction, and induces the algae to accumulate additional lipids resulting in a second average lipid content that averages at least 50% more than the first %.

Step 103 comprises measuring a lipid concentration of the algae while being under the stress inducing environmental condition. Step 104 comprises harvesting lipids from more than 50% but not all of the algae when the lipid concentration measured is above a predetermined minimum lipid limit. Step 105 comprises adding fresh medium to the bioreactor medium having the algae not involved in the harvesting therein, and repeating the method, wherein the algae not involved in harvesting serves as a source for new algae growth in the bioreactor medium after the adding. Thus, after sufficient lipid accumulation has taken place, some but not all the cells are harvested and lipids extracted. For example, in one particular embodiment 90% of the cells can be harvested, with the remaining 10% returned to full growth conditions and combined with 90% fresh medium (i.e., diluting the 10% of the algae not harvested and letting them grow to the maximum density again), and the process repeated.

As used herein “stops cell reproduction” is defined to include cells that might provide a division or two, especially if they are already in the process of splitting upon initiation of the stress inducing environmental condition. Once the cells are moved into the lipid-enhancement phase, the cells are no longer provided what they need for dividing, and they will thus stop dividing. The conditions in step 102 keep the cells alive and allows the degradation of chloroplasts into lipids, as well as synthesis of additional lipids.

As used herein “degradation of membrane lipids of the algae” is defined to include lipids that are altered so they are no longer physically assembled into the cell membrane, that involve chemical alterations to the molecules to allow them to be packed into a centralized lipid globule. Regarding “accumulating additional lipids”, the stress inducing environmental condition(s) can increase the lipid content in species of algae from an average of 25% to 50% of the total cell biomass. Selected strains have been found to be able to be forced to accumulate 50% of the total cell weight as lipid, with some species as high as about 70% lipid by weight.

Example techniques for triggering lipid-enhanced accumulating techniques that inhibit cellar division (individually or used in combination) include, but are not limited to:
1. Adding a plant hormone, such as gibberellic acid, at an exemplary concentration of about 25 μM±50% to the algal cells in the bioreactor medium during the late exponential stage of growth. As defined herein, the “late exponential stage of growth” refers to the point in the growth curve that cell numbers increase less than 10% on consecutive days.
2. Adding a plant hormone, such as abscisic acid, at an exemplary concentration of about 25 μM±50% to the algal cells during the late exponential stage of growth. Other example hormones include cytokinin.
3. Growing a liquid culture of bacteria Pseudomonas spp. in nutrient broth until it reaches stationary phase. As defined herein, the “stationary phase” refers to the point in the growth curve that cell numbers do not increase on consecutive days. The bacteria can be filtered out and the filtrate used (about 1:10 to 1:30 v:v ratio-filtrate: algal cultures) to add to algal cells in the bioreactor medium during the late exponential stage of growth.
4. Lowering the nitrate and ammonia concentration in the algae cultures in the bioreactor medium below about 0.1 mM dilution±50% with nitrogen-free or nitrogen-limited (below about 0.1 mM±50%) water.
5. Adding a compound that disrupts photosynthesis ((e.g. 3-(3,4-dichlorophenyl)-1,1-dimethylurea or some other herbicide) at a final concentration, such as 10 μg/L±50% to the algal cells in the bioreactor medium during the late exponential stage of growth.
6. Adding compounds that microtubules (the subcellular structures that are responsible of cell division), such as Colchicine at a final concentration, such as about 10 μg/L±50% to the algal cells in the bioreactor medium during the late exponential stage of growth.
7. Grow a liquid culture of the cyanobacteria, such as Lyngbya, Phormidium, Osillatoria or other species, until it reaches the stationary phase. Filter out the cyanobacteria and use the filtrate (e.g., about 1:10 to 1:30 v:v ratio-filtrate: algal cultures) added to algal cells in the bioreactor medium during the late exponential stage of growth.
   Exemplary techniques for triggering lipid-physical stimuli (individually or in combination) include, but are not limited to:
1. During late exponential growth phase, raise the temperature in the bioreactor medium by about 1 to 5° C., such as 3° C.
2. During late exponential growth phase, lower the temperature in the bioreactor medium by about 1 to 5° C., such as 3° C.
3. During late exponential growth phase, block essentially all light sources to the bioreactor medium that stimulate photosynthesis for 24 to 72 hours, such as 48 hours.
4. During late exponential growth phase, raise the pH in the bioreactor medium by about 1 log unit±50% by the addition of bases such as sodium hydroxide.
5. During late exponential growth phase, lower the pH in the bioreactor medium by 2 log units±50% by addition of acids such as hydrochloric acid.

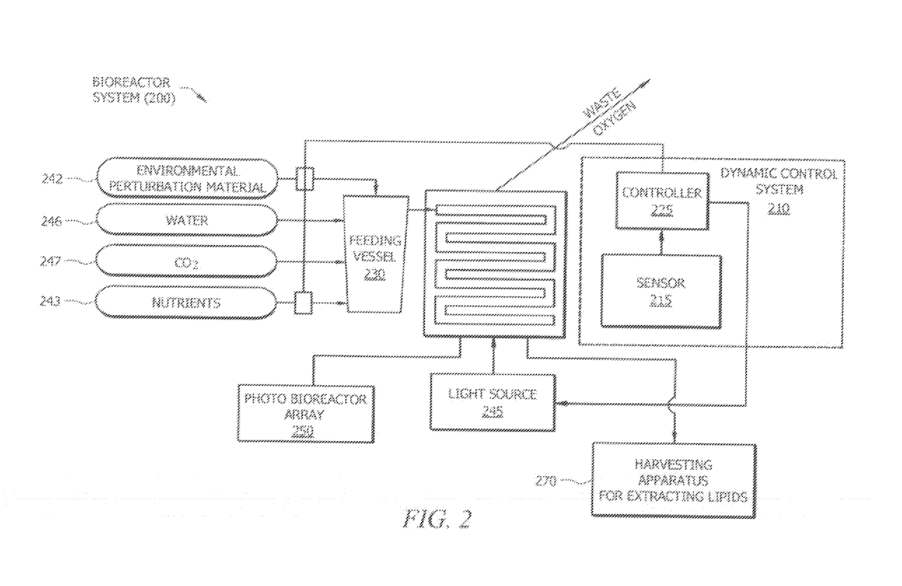

FIG. 2 is a block diagram of an exemplary bioreactor system 200 for generating enhanced lipid production from algae, including a dynamic control system 210, according to an embodiment of the invention. Bioreactor system 200 includes a feeding vessel 230, a photo bioreactor array 250, and a dynamic control system 210 that includes at least one sensor 215 and a controller 225. Controller 225 is shown coupled to adjust the amounts of an environmental perturbation material 242, nutrients 243 added to the bioreactor medium, and the output of light source 245. Feeding vessel 230 is shown receiving environmental perturbation material 242, water (e.g., recycled water) 246, and carbon dioxide (CO₂) 247, where the output of feeding vessel 230 is coupled to photo bioreactor array 250 that includes the bioreactor medium. CO₂ 247 is generally provided in a level up to 20 vol. %.

Sensors 215 can be provided for measuring parameters such as pH, carbon dioxide level, temperature, light quantity, and lipid content in the algae. Lipid concentration can be sensed and thus quantified using epifluorescent microscopy enabled by the addition of a lipid stain to the photo bioreactor medium. As disclosed above, in one particular embodiment 90% of the cells in the reactor are harvested by directing them from photo bioreactor array 250 to a harvesting apparatus for extracting lipids 270. Fresh medium is then added to the bioreactor medium, with the remaining (e.g., 10%) of the cells serving as the source for new cell growth in the bioreactor medium, which allows bioreactor system 200 to be able to provide continuous lipid output while being free from the need for algae additions during production required for conventional bioreactor systems.

An example method is now disclosed. Algae are grown using a medium that is complete with all macronutrients and micronutrients, aerated for mixing, with ample light. The algae are monitored at least daily and it is determined when the growth rate is reduced, indicating that cells have exhausted a vital nutrient or have become limited by light. At this time, most but not all (e.g., 90%) of the algae are harvested and placed into a treatment vat and a growth-inhibiting compound is added to the medium and/or the physical conditions are altered, and the bioreactor is monitored for lipid accumulation. For example, the cells can be examined with an epifluorescent microscope using a lipid stain, such as Nile Red. The lipids are harvested/extracted when maximum lipid accumulation has occurred. One extraction method is wet extraction with 100% ethanol applied to the cells that are removed from the bioreactor. Other extraction methods may also be used.

The process is generally a repetitive process. For example, 90% of the cells can be harvested, with the remaining 10% returned to full growth conditions and combined with 90% fresh medium (i.e., diluting the 10% of the algae not harvested and letting them grow to the maximum density again), and the process is repeated.

As known in the art, algae lipids produced by disclosed methods can be converted into to biodiesel (fatty acid methyl esters-FAME). For example, separation or extraction processes can be used.

While various embodiments of the invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the invention should not be limited by any of the above described embodiments. Rather, the scope of the invention should be defined in accordance with the following claims and their equivalents.

Although embodiments of the invention have been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

I claim:

1. A method for stimulating enhanced lipid accumulation by algae, comprising:
growing said algae in a bioreactor medium including macronutrients and micronutrients having light reaching said bioreactor medium, wherein said algae have an average lipid content that averages a first % of total cell biomass, and
initiating a stress inducing environmental condition that keep said algae alive, stops cell reproduction, and induces said algae to accumulate additional lipids resulting in a second average lipid content that averages at least 50% more than said first %.

2. The method of claim 1, wherein said first % is at least 25% and said second average lipid content is at least 50%.

3. The method of claim 1, wherein said stress inducing environmental condition comprises adding a plant hormone to said bioreactor medium during a late exponential stage of growth of said algae.

4. The method of claim 1, wherein said stress inducing environmental condition comprises adding a bacteria to said bioreactor medium.

5. The method of claim 1, wherein said stress inducing environmental condition comprises lowering a nitrate and ammonia concentration in said bioreactor medium.

6. The method of claim 1, wherein said stress inducing environmental condition comprises adding a compound to said bioreactor medium during a late exponential stage of growth of said algae.

7. The method of claim 1, wherein said stress inducing environmental condition comprises raising or lowering a temperature of said bioreactor medium by 1 to 5° C. during a late exponential growth phase of said algae.

8. The method of claim 1, wherein said stress inducing environmental condition comprises blocking essentially all light sources to said bioreactor medium that stimulate photosynthesis for at least 24 hours during a late exponential growth phase of said algae.

9. The method of claim 1, wherein said inducing environmental condition comprises raising or lowering a pH in said bioreactor medium by at least 1 log unit during a late exponential growth phase of said algae.

10. The method of claim 1, further comprising:
measuring a lipid concentration of said algae while being under said stress inducing environmental condition;
harvesting lipids from more than 50% but not all of said algae when said lipid concentration measured is above a predetermined minimum lipid limit, and
and adding fresh medium to said bioreactor medium having said algae not involved in said harvesting therein and repeating said method, wherein said algae not involved in said harvesting serves as a source for new algae growth in said bioreactor medium after said adding.

11. A bioreactor system for generating enhanced lipid production from algae, comprising:
a photo bioreactor array including a bioreactor medium for said algae therein including nutrients having light reaching said bioreactor medium, wherein said algae have an average lipid content that averages a first % of total cell biomass, and
a dynamic control system for initiating and maintaining a stress inducing environmental condition, wherein said dynamic control system comprises:
at least one sensor coupled to sense at least one parameter associated with said bioreactor medium, and
a controller coupled to receive a sensing signal from said sensor, wherein said controller is coupled to control at least one of an output of said light source, a concentration of said nutrients added to said bioreactor medium, a concentration nutrients added to said bioreactor medium, and an amount of said environmental perturbation material added to said bioreactor medium,
wherein said stress inducing environmental condition keeps said algae alive, stops cell reproduction, and induces said algae to accumulate additional lipids resulting in a second average lipid content that averages at least 50% more than said first %.

12. The bioreactor system of claim 11, wherein said dynamic control system is operable for measuring a lipid concentration of said algae while being under said stress inducing environmental condition.
